(19) United States
(12) Patent Application Publication
(10) Pub. No.: US 2016/0100848 A1
(43) Pub. Date: Apr. 14, 2016

(54) AUXILIARY JIG FOR TRAPEZIOMETACARPAL JOINT ARTHROPLASTY

(71) Applicant: NATIONAL CHENG KUNG UNIVERSITY, TAINAN CITY (TW)

(72) Inventors: Wei-jr LIN, Tainan city (TW); I-ming JOU, Tainan city (TW); Che-chia HSU, Tainan city (TW); Fong-chin SU, Tainan city (TW)

(21) Appl. No.: 14/510,588

(22) Filed: Oct. 9, 2014

(51) Int. Cl. A61B 17/17

(52) U.S. Cl. CPC A61B 17/1739; A61B 2017/1782

(57) ABSTRACT

An auxiliary jig for a trapeziometacarpal joint arthroplasty is disclosed. The jig has a body, two grip portions, and a positioning portion. The positioning portion has a plurality of positioning holes so that pins can be fixed on the first metacarpal bone through the positioning holes. The jig can provide accurate positioning operations for cutting and drilling during a trapeziometacarpal joint arthroplasty.

### FIELD OF THE INVENTION

The present invention relates to an auxiliary jig, and more particularly to an auxiliary jig for a trapeziometacarpal joint arthroplasty.

### BACKGROUND OF THE INVENTION

Osteoarthritis is a disease that affects the joints. When the intra-articular cartilage of the joints is damaged or worn, it causes osteoarthritis, and thus causes joint stiffness, pain, and loss of function. Osteoarthritis commonly occurs in hip joints, knee joints, spinal joints, and joints of the hand (such as carpometacarpal joints). Among these, trapeziometacarpal (TMC) joint arthritis is one of the most important issues in the degenerative arthritis disease of the upper limbs. When medicaments or injections are not effective in relieving pain and can't further deal with the degenerative arthritis of the TMC joint, the injured TMC joint can be replaced by arthroplastic surgery, thereby not only reducing pain from arthritis, but also maintaining TMC joint activities and the functions of the hand.

The process of a traditional surgery for replacing an artificial TMC joint is firstly to crosscut a wound from the root of the thumb, then release the attachment of the abductor pollicis longus (APL), open the joint capsule of the TMC joint, and abscise the damaged TMC joint surface at the proximal end of the first metacarpal to form an incision. Then, a portion of an implant is inserted into a bone marrow cavity of the first metacarpal from the incision at the proximal end of the first metacarpal. Thereafter, a surgical suture is used to reattach the APL tendon, followed by suturing the wound.

However, a metacarpal bone is cut or drilled without an assisted positioning tool during a trapeziometacarpal joint arthroplasty, and the position for cutting or drilling is not accurate, so that the metacarpal bone could require further cutting or drilling, and the time required to perform the surgery would be increased.

As a result, it is necessary to provide an auxiliary jig for the trapeziometacarpal joint arthroplasty to solve the problems existing in the conventional technologies, as described above.

### SUMMARY OF THE INVENTION

A primary object of the present invention is to provide an auxiliary jig which are fixed on the metacarpal bone by pins through positioning holes for trapeziometacarpal joint arthroplasty, so that the metacarpal bone can be cut accurately by the auxiliary jig.

To achieve the above object, the present invention provides an auxiliary jig for trapeziometacarpal joint arthroplasty, which comprises a body having an inner surface for abutting against a metacarpal bone and an outer surface in opposite the inner surface; two grip portions provided on two sides of the outer surface of the body, respectively; and a positioning portion extended from an end of the body, and the positioning portion having a plurality of positioning holes, and the body being fixed on the metacarpal bone by fixing a plurality of pins on the metacarpal bone through the positioning holes.

In one embodiment of the present invention, each of the grip portions has a concave curved surface.

In one embodiment of the present invention, the positioning holes are spaced from each other and arranged in a straight line.

In one embodiment of the present invention, the positioning portion has a tapered end.

In one embodiment of the present invention, the body is formed with a first linear through hole, and a blade inserts to cut the articular surface of metacarpal bone through the first linear through hole.

In one embodiment of the present invention, the body is formed with a second linear through hole, and the first linear through hole is perpendicular to the second linear through hole.

In one embodiment of the present invention, a fin is extended from the inner surface for abutting against an articular surface of the metacarpal bone.

In one embodiment of the present invention, the fin has a tapered end.

In one embodiment of the present invention, the fin has a third linear through hole close to the inner surface of the body.

In one embodiment of the present invention, a drilling positioning plate is projected from a distal end of the body, and the drilling positioning plate is formed with at least one through hole, so that a drill drills the metacarpal bone through the through hole.

As stated above, the pins are fixed on the metacarpal bone through the positioning holes, so that the metacarpal bone can be cut accurately by the auxiliary jig. The auxiliary jig can provide accurate positioning operations for cutting and drilling during trapeziometacarpal joint arthroplasty to avoid cutting the metacarpal bone again. Thus, the time required to perform the surgery can be decreased.

### DESCRIPTION OF THE DRAWINGS

FIG. 1 is a perspective view of an auxiliary jig for trapeziometacarpal joint arthroplasty according to a first preferred embodiment of the present invention;

FIG. 2 is a front view of an auxiliary jig for trapeziometacarpal joint arthroplasty according to a first preferred embodiment of the present invention;

FIG. 3 is a side view of an auxiliary jig for trapeziometacarpal joint arthroplasty according to a first preferred embodiment of the present invention;

FIG. 4 is a vertical view of an auxiliary jig for trapeziometacarpal joint arthroplasty according to a first preferred embodiment of the present invention;

FIG. 5 is a perspective view of an auxiliary jig for trapeziometacarpal joint arthroplasty according to a second preferred embodiment of the present invention;

FIG. 6 is a front view of an auxiliary jig for trapeziometacarpal joint arthroplasty according to a second preferred embodiment of the present invention;

FIG. 7 is a side view of an auxiliary jig for trapeziometacarpal joint arthroplasty according to a second preferred embodiment of the present invention; and

FIG. 8 is a vertical view of an auxiliary jig for trapeziometacarpal joint arthroplasty according to a first preferred embodiment of the present invention.

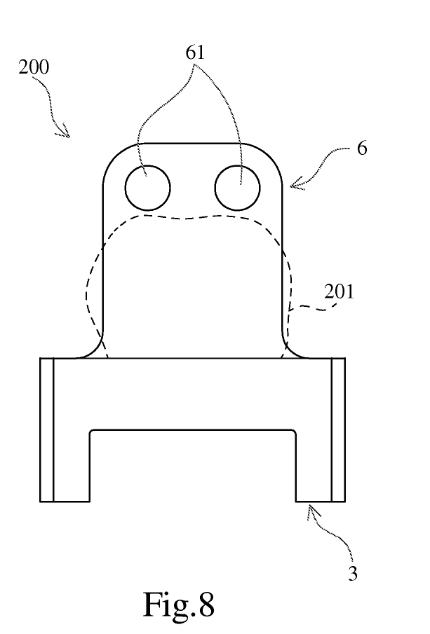

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

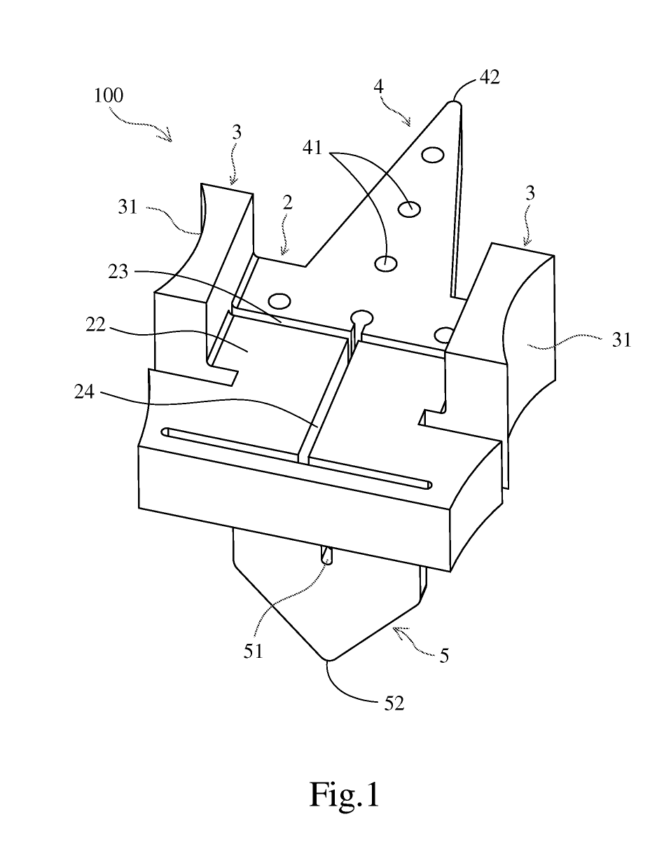

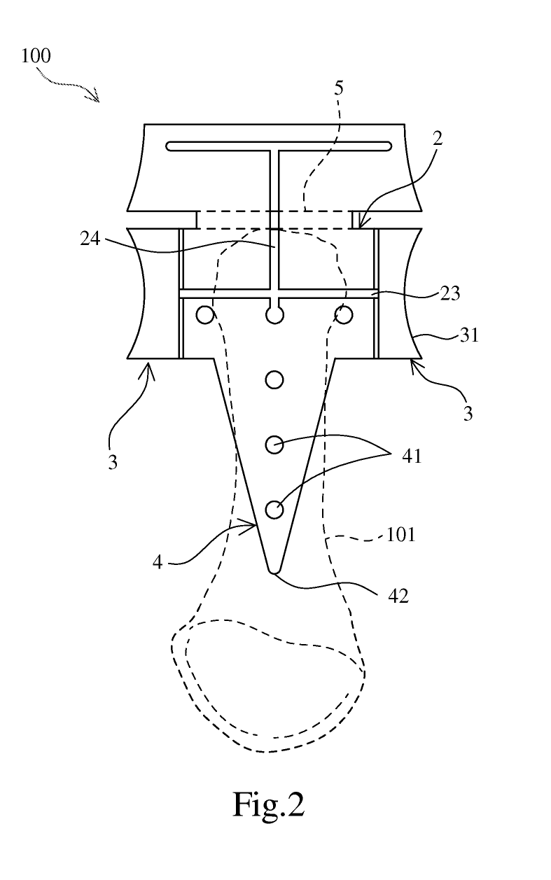

Referring now to FIGS. 1 and 2, wherein an auxiliary jig for trapeziometacarpal joint arthroplasty according to a first preferred embodiment of the present invention is illustrated. As shown, the auxiliary jig 100 is positioned on a metacarpal bone 101 to cut the metacarpal bone 101, and the auxiliary jig 100 comprises a body 2, two grip portions 3, a positioning portion 4, and a fin 5. The body 2, the grip portions 3, the positioning portion 4, and the fin 5 are integrated and made of biocompatible metarial, such as titanium, titanium alloy, polymer, cobalt-chromium-molybdenum alloy, or stainless steel, but are not limited thereto. The detailed structure of each component, assembly relationships and principle of operation in the present invention will be described in detail hereinafter.

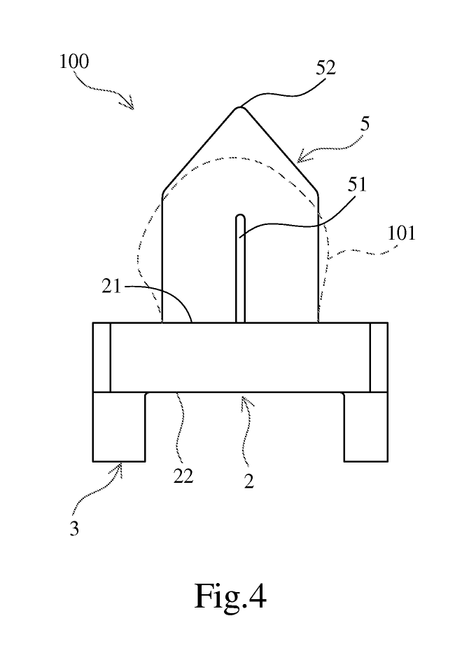

Referring now to FIGS. 1, 2, and 4, in the first preferred embodiment of the present invention, the body 2 has an inner surface 21 for abutting against the metacarpal bone 101, an outer surface 22 opposite the inner surface 21, a first linear through hole 23 through the inner surface 21 and the outer surface 22, and a second linear through hole 24. The second linear through hole 24 is perpendicular to the first linear through hole 23 through the inner surface 21 and the outer surface 22, and a blade inserts to cut the articular surface of the metacarpal bone 101 through the first linear through hole 23 or the second linear through hole 24 (shown FIG. 2) in the first preferred embodiment. In other embodiments, the first linear through hole 23 and the second linear through hole 24 are independent of each other, but the present invention is not limited thereto.

Referring now to FIGS. 1 and 2, in the first preferred embodiment of the present invention, the grip portions 3 are provided on two sides of the outer surface 22 of the body 2, respectively. Wherein each of the grip portions 3 has a concave curved surface 31 for touching with a finger steady, and the friction and the stability of the grip portions 3 can be improved.

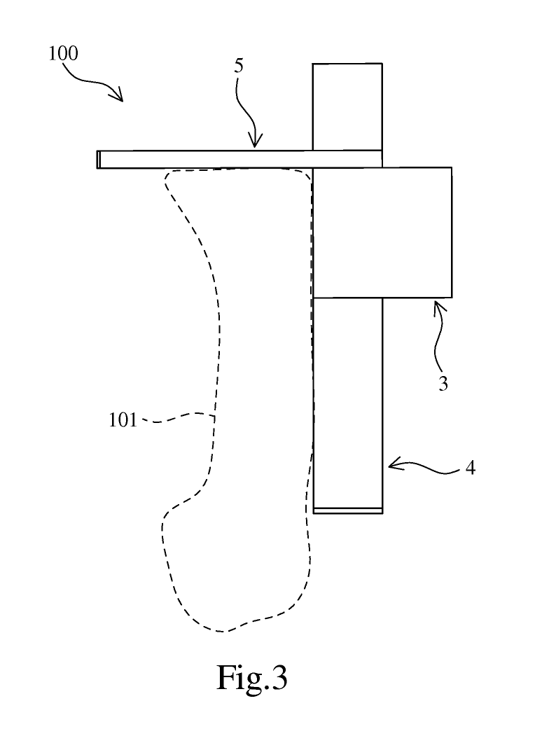

Referring now to FIGS. 1, 2, and 3, in the first preferred embodiment of the present invention, the positioning portion 4 is extended from an end of the body 2, and the metacarpal bone 101 can be connected to the positioning portion 4. The positioning portion 4 has a plurality of positioning holes 41 for fixing a plurality of pins on the metacarpal bone 101 so that the body 2 can be positioned on the metacarpal bone 101 and the metacarpal bone 101 can be cut accurately. The positioning holes 4 are spaced from each other and arranged in a straight line in the first preferred embodiment, and the positioning portion has a tapered end 42 for aligning to the center line of the metacarpal bone 101 so that the positioning holes 41 are positioned on the metacarpal bone 101 quickly before fixing pins.

Referring now to FIGS. 1, 3, and 4, in the first preferred embodiment of the present invention, the fin 5 is extended from the inner surface 21 of the body 2 for abutting against the articular surface of the metacarpal bone 101. The fin 5 has a third linear through hole 51 close to the inner surface 21 of the body 2 and a tapered end 52. The blade can cut the metacarpal bone through the third linear through hole 51, and the tapered end 52 can be aligned to the center line of the metacarpal bone 101.

Referring now to FIGS. 1 to 4, first, the auxiliary jig 100 is placed on the metacarpal bone 101 along the center line of the metacarpal bone 101, and the positioning holes 41 and the tapered end 42 are aligned to the center line, so that the fin 5 is attached to the articular surface of the metacarpal bone 101. Then pins are fixed on the metacarpal bone 101 through the positioning holes 41, and the blade inserts to cut the articular surface of the metacarpal bone 101 through the first linear through hole 23. In addition, the blade also inserts to cut the metacarpal bone 101 through the second linear through hole 24 and the third linear through hole 51 accurately.

As stated above, the pins are fixed on the metacarpal bone 101 through the positioning holes 41, so that the metacarpal bone 101 can be cut accurately by the auxiliary jig 100. The auxiliary jig 100 can provide accurate positioning operations for cutting and drilling during a trapeziometacarpal joint arthroplasty to avoid cutting the metacarpal bone 101 again. Thus, the time required to perform the surgery can be decreased.

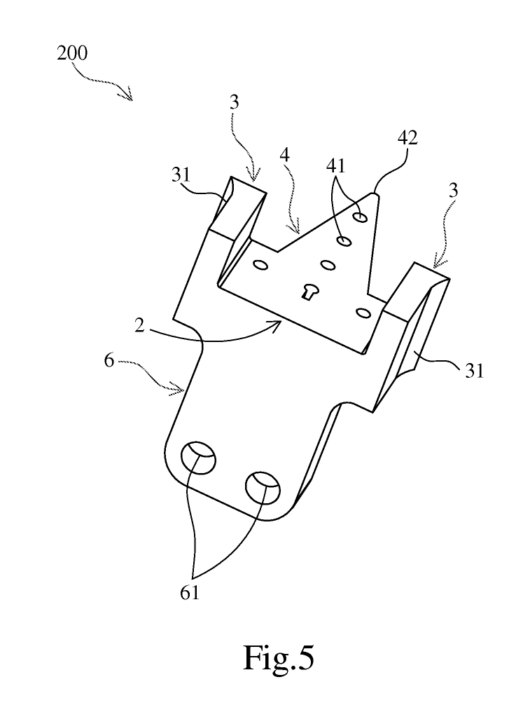

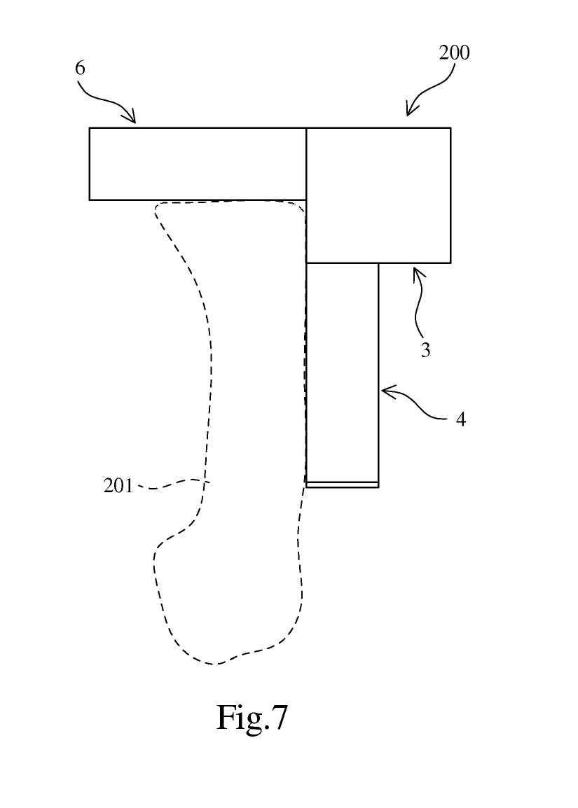

Referring now to FIGS. 5, 6, and 7, an auxiliary jig for trapeziometacarpal joint arthroplasty according to a second preferred embodiment of the present invention is illustrated, and is similar to the first preferred embodiments. The auxiliary jig 200 also comprises a body 2, two grip portions 3, and a positioning portion 4, so that the second preferred embodiment uses terms or numerals similar to the first embodiment. As shown, the difference of the second preferred embodiment is that the body 2 only has an inner surface 21 and an outer surface 22. The auxiliary jig 200 is positioned on a metacarpal bone 201 to drill the metacarpal bone 201, and the auxiliary jig 200 further comprises a drilling positioning plate 6. The body 2, the grip portions 3, and the positioning portion 4 are integrated and made of biocompatible material, such as titanium, titanium alloy, polymer material, cobalt-chromium-molybdenum alloy, or stainless steel, but are not limited thereto. The detailed structure of each component, assembly relationships, and principles of operation in the present invention will be described in detail hereinafter.

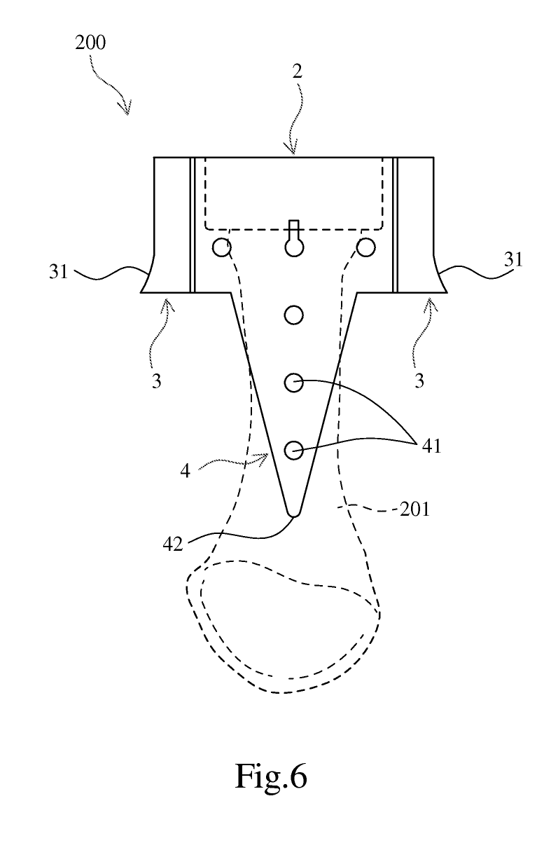

Referring now to FIGS. 5 and 6, in the second preferred embodiment of the present invention, the drilling positioning plate 6 is projected from a distal end of the body 2, and the drilling positioning plate 6 is formed with at least one through hole 61. There are two through hole 61 for drilling the metacarpal bone 201 in the second preferred embodiment. In other embodiments the drilling positioning plate 6 is formed with more than two through holes 61, but the present invention is not limited thereto.

Referring now to FIGS. 5 and 6, after the cutting process of the first preferred embodiment, the auxiliary jig 200 is placed on the metacarpal bone 201, and the pins are inserted through the auxiliary jig 200. The drill is drilled into the metacarpal bone 201, so that one or more holes are drilled in order to insert an artificial implant.

As stated above, the pins are fixed on the metacarpal bone 201 through the positioning holes 41, so that the metacarpal bone 201 can be drilled accurately by the auxiliary jig 200. The auxiliary jig 200 can provide accurate positioning operations for drilling during trapeziometacarpal joint arthroplasty to avoid drilling the metacarpal bone 201 again. Thus, the time required to perform the surgery can be decreased.

The present invention has been described with two preferred embodiment thereof and it is understood that many changes and modifications to the described embodiments can be carried out without departing from the scope and the spirit of the invention that is intended to be limited only by the appended claims.

What is claimed is:

1. An auxiliary jig for trapeziometacarpal joint arthroplasty, comprising:
a body having an inner surface for abutting against a metacarpal bone and an outer surface opposite the inner surface;
two grip portions provided on two sides of the outer surface of the body, respectively; and
a positioning portion extended from an end of the body, the positioning portion having a plurality of positioning holes, and the body being fixed on the metacarpal bone by fixing a plurality of pins on the metacarpal bone through the positioning holes.

2. The auxiliary jig according to claim 1, wherein each of the grip portions has a concave curved surface.

3. The auxiliary jig according to claim 1, wherein the positioning holes are spaced from each other and arranged in a straight line.

4. The auxiliary jig according to claim 1, wherein the positioning portion has a tapered end.

5. The auxiliary jig according to claim 1, wherein the body is formed with a first linear through hole, and a blade inserts to remove an articular surface of the metacarpal bone through the first linear through hole.

6. The auxiliary jig according to claim 5, wherein the body is formed with a second linear through hole, and the first linear through hole is perpendicular to the second linear through hole.

7. The auxiliary jig according to claim 1, further comprising: a fin extended from the inner surface for abutting against an articular surface of the metacarpal bone.

8. The auxiliary jig according to claim 7, wherein the fin has a tapered end.

9. The auxiliary jig according to claim 8, wherein the fin has a third linear through hole close to the inner surface of the body.

10. The auxiliary jig according to claim 1, further comprising: a drilling positioning plate projected from a distal end of the body, and the drilling positioning plate is formed with at least one through hole, so that a drill drills the metacarpal bone through the through hole.
